(19) United States
(12) Patent Application Publication
(10) Pub. No.: US 2011/0071391 A1
(43) Pub. Date: Mar. 24, 2011

(54) BIOPSY MARKER DELIVERY DEVICE WITH POSITIONING COMPONENT

(76) Inventor: Trevor W.V. Speeg, Williamsburg, OH (US)

(21) Appl. No.: 12/565,968

(22) Filed: Sep. 24, 2009

(51) Int. Cl. A61B 6/00; B29C 45/14

(52) U.S. Cl. 600/431; 264/271.1

(57) ABSTRACT

A biopsy marker delivery device are described. The delivery device can include a relatively flexible hollow tube, a pushing member such as a push rod disposed for sliding with the tube, and at least one marker disposed in the tube. A positioning component is described and shown disposed around the hollow tube.

### CROSS REFERENCE TO RELATED APPLICATIONS

This application cross references and incorporates by reference the following commonly assigned applications: U.S. patent application Ser. No. 12/196,301 filed Aug. 22, 2008; U.S. patent application Ser. No. 12/563,360 filed Sep. 21, 2009; U.S. patent application Ser. No. 12/365,390 filed Feb. 4, 2009; and U.S. patent application Ser. No. 12/406,135 filed Mar. 18, 2009.

### BACKGROUND

Biopsy samples have been obtained in a variety of ways in various medical procedures using a variety of devices. An exemplary biopsy device is the MAMMOTOME® brand device from Ethicon Endo-Surgery, Inc. of Cincinnati, Ohio. Biopsy devices may be used under stereotactic guidance, ultrasound guidance, MRI guidance, or otherwise.

Further exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2007/0118048, entitled “Remote Thumbwheel for a Surgical Biopsy Device,” published May 24, 2007; U.S. Provisional Patent Application Ser. No. 60/869,736, entitled “Biopsy System,” filed Dec. 13, 2006; U.S. Provisional Patent Application Ser. No. 60/874,792, entitled “Biopsy Sample Storage,” filed Dec. 13, 2006; and U.S. Non-Provisional patent application Ser. No. 11/942,785, entitled “Revolving Tissue Sample Holder for Biopsy Device,” filed Nov. 21, 2007. The disclosure of each of the above-cited U.S. patents, U.S. patent application Publications, U.S. Provisional patent applications, and U.S. Non-Provisional patent application is incorporated by reference herein.

In some settings, it may be desirable to mark the location of a biopsy site for future reference. For instance, one or more markers may be deposited at a biopsy site before, during, or after a tissue sample is taken from the biopsy site. Exemplary marker deployment tools include the MAMMOMARK®, MICROMARK®, and CORMARK® brand devices from Ethicon Endo-Surgery, Inc. of Cincinnati, Ohio. Further exemplary devices and methods for marking a biopsy site are disclosed in U.S. Pub. No. 2005/0228311, entitled “Marker Device and Method of Deploying a Cavity Marker Using a Surgical Biopsy Device,” published Oct. 13, 2005; U.S. Pat. No. 6,996,433, entitled “Imageable Biopsy Site Marker,” issued Feb. 7, 2006; U.S. Pat. No. 6,993,375, entitled “Tissue Site Markers for In Vivo Imaging,” issued Jan. 31, 2006; U.S. Pat. No. 7,047,063, entitled “Tissue Site Markers for In Vivo Imaging,” issued May 16, 2006; U.S. Pat. No. 7,229,417, entitled “Methods for Marking a Biopsy Site,” issued Jun. 12, 2007; U.S. Pat. No. 7,044,957, entitled “Devices for Defining and Marking Tissue,” issued May 16, 2006; U.S. Pat. No. 6,228,055, entitled “Devices for Marking and Defining Particular Locations in Body Tissue,” issued May 8, 2001; and U.S. Pat. No. 6,371,904, entitled “Subcutaneous Cavity Marking Device and Method,” issued Apr. 16, 2002. The disclosure of each of the above-cited U.S. patents and U.S. patent application Publications is incorporated by reference herein.

It may be desirable to deploy markers from a cannula type deployer into the biopsy site, such as a flexible tubular deployer. The marker should not unintentionally fall out of the deployer, and the force to deploy the marker should not be excessive. Further, the tubular deployer should not advance further within the biopsy device than intended.

### SUMMARY

In one non-limiting aspect, the present invention provides a biopsy marker deployer comprising a tube carrying at least one biopsy marker, and inner pushing member such as push rod. The push rod is disposed within the outer tube and is advanceable within the tube to urge the marker out of the deployer.

Applicant has recognized the desirability of avoiding “over advancing” the deployer, and particularly the deployer tip, into the associated biopsy device when deploying markers through a biopsy device.

Applicant has further recognized the desirability of providing a feature on the outside of the deployer tube (or shaft), the feature disposed a predetermined distance from the distal tip of the deployer.

According to one aspect of the present invention, a method is provided. The method may include the steps of obtaining a hollow tube having a sidewall, a proximal end, a distal end, and an internal lumen; forming an endpiece in place in the distal end of the hollow tube to close the distal end of the tube; forming a component extending around an outside surface of the hollow tube, wherein the step of forming the component comprises positioning the component along the tube at a predetermined distance from the endpiece between proximal end of the hollow tube and the distal end of the hollow tube; and disposing at least one biopsy marker in the internal lumen.

The method may further comprise forming an opening in the side wall of the tube between the distal end of the hollow tube and the component, the opening providing an exit through which a marker may be deployed.

In another aspect of the present invention, a biopsy marker delivery device is provided for deploying biopsy markers. The device may include: a cannula having an internal lumen extending from a proximal end of the cannula to the distal end of the cannula, and a marker exit formed in a sidewall of the cannula proximal to the distal end of the cannula; a distal tip closing the distal end of the cannula; a component disposed on the outer surface of the cannula sidewall, the component disposed at a position along the length of the cannula between the proximal end of the cannula and the marker exit formed in the sidewall of the cannula, and the component being sized and shaped to limit the depth to which the cannula can be inserted within a biopsy instrument; and at least one marker disposed within the internal lumen of the cannula.

The component may be annular in shape and may be disposed at a position along the length of the cannula at a predetermined distance from the distal tip. The component may be overmolded on the cannula, and fixed with respect to the cannula. The component may further include an proximally extending tab circumferentially aligned with the marker exit port.

The component may be positioned a predetermined distance between a grip (disposed at or near the proximal end of the cannula) and at least one of the marker exit and the tip. The component may have an annular body having an axial length of less than about 0.5 inch and an axially extending tab, the tab extending proximally from the annular body, and the tab having a length at least about twice the axial length of the annular body.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of a marker delivery device of the type illustrated in U.S. patent application Ser. No. 12/196,301 filed Aug. 22, 2008;

FIG. 2 depicts a cross-sectional view of a distal portion of a marker delivery device of the type illustrated in U.S. patent application Ser. No. 12/196,301 filed Aug. 22, 2008.

FIG. 3 depicts a marker being deployed from a deployer and through a lateral tissue receiving port in a biopsy needle to mark a biopsy site, such as illustrated in U.S. patent application Ser. No. 12/196,301 filed Aug. 22, 2008.

FIG. 4 depicts a portion of a marker deployment tube is shown cutaway to illustrate the inner diameter of the tube is generally smooth, and to reveal a member, such as a push rod, the push rod having a surface feature effective for reducing the contact surface area between a portion of the push rod disposed within the deployment tube and the inner surface of the deployment tube, and FIG. 4 showing a push rod having a surface finish and surface roughness different than those of the inner diameter of the tube, the push rod shown having a plurality of generally longitudinally extending ribs having peaks elevated above relatively lower elevation depressions.

FIG. 5. Depicts a cross-section of the pushing member and illustrating the peaks of the elevated portions of the longitudinally extending ribs in relation to the diameter of the pushing member and in relation to the recessed portions of the outer surface of the push rod.

FIG. 6 depicts a cross-section of the deployer with the pushing member shown disposed within the deployer tube, and illustrating the circumferential peak to peak spacing of adjacent longitudinally extending ribs can be greater than the radial height of the ribs.

FIG. 7 illustrates a pushrod having a relatively rigid proximal portion 18A (such as stiffened by a metal sleeve) and a relatively flexible distal portion 18B comprising a plurality of longitudinally extending ribs.

FIG. 8 illustrates a plurality of ring like ribs providing spaced apart raised surfaces disposed on the outer surface of a push rod, adjacent ring like ribs spaced longitudinally from one another along a portion of the push rod disposed within the deployment tube.

FIG. 9 illustrates an embodiment having a surface feature on the inside surface of the cannula lumen.

FIG. 10 is a schematic illustration showing a marker delivery device according to one embodiment of the present invention, and showing a component, such as a positioning component having a generally annular body and a proximally extending member, the component positioned at predetermined distance from the tip of the delivery device, and the component positioned at a predetermined distance with respect to a side marker exit.

FIG. 11 is an enlarged schematic illustration of a distal portion of the device of FIG. 10, FIG. 11 illustrating a distal tip and a side marker exit port disposed proximally of the distal tip, the exit port having a proximal portion and a relatively larger distal portion.

FIG. 12 is a schematic illustration of the component shown in FIG. 10 positioned with respect to an opening in a biopsy device, FIG. 12 illustrating an alignment feature, such as a distally extending male alignment feature extending from the annular body of the positioning component for engagement with a female alignment feature, such as an alignment groove, formed in portion of a biopsy device.

FIG. 13 is a schematic illustration of a biopsy marker delivery device according to an embodiment of the present invention, FIG. 13 providing a top view of the biopsy marker delivery device and illustrating alignment of a positioning component with the biopsy marker exit.

FIG. 14 is a schematic illustration of the biopsy marker device of FIG. 13, FIG. 13 providing a side view of the biopsy device of FIG. 13 and illustrating predetermined spacing of a positioning component with respect to the distal tip of the device, such as where the positioning component and the distal tip are molded in place with respect to the cannula of the delivery device in the same molding operation.

FIG. 15 is a schematic illustration of a biopsy marker delivery device according to an embodiment of the present invention positioned for insertion into a proximal opening in a proximal end of a biopsy device.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

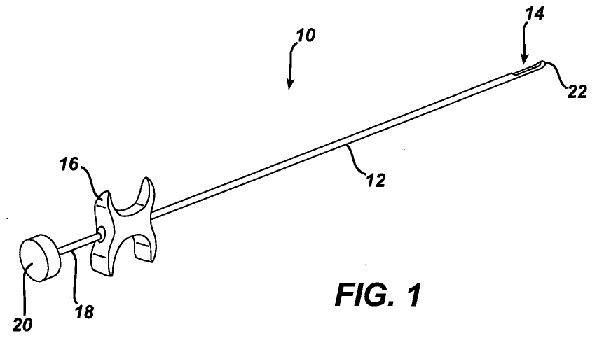

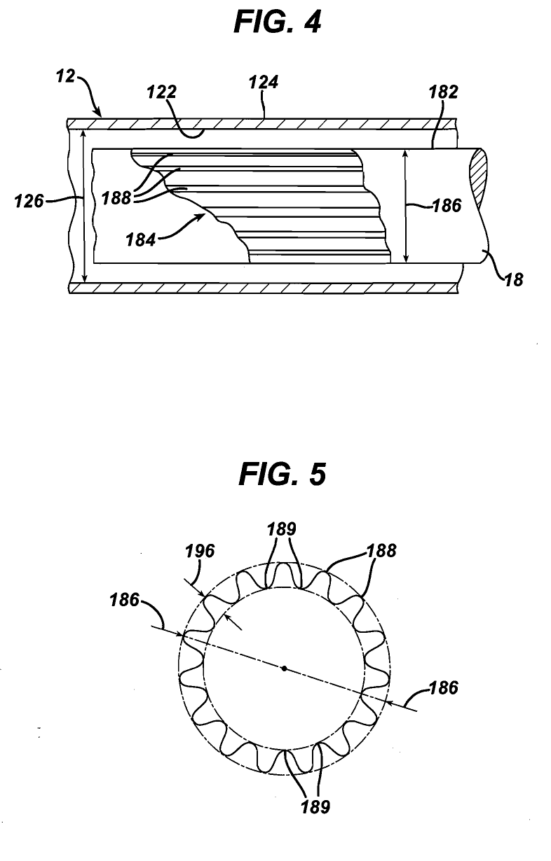

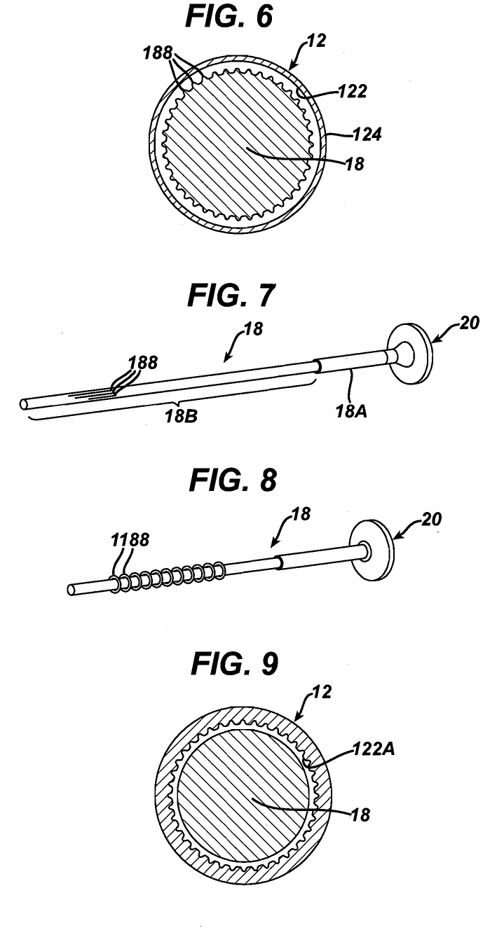

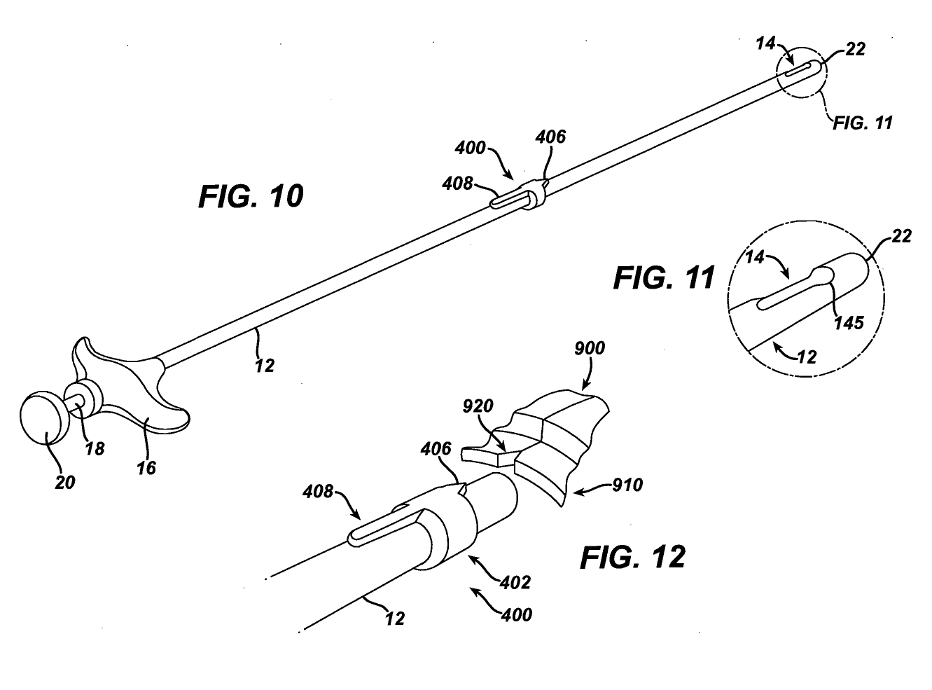

FIGS. 1-3 illustrate a marker delivery device 10 of the type illustrated in copending, commonly assigned U.S. patent application Ser. No. 12/196,301 filed Aug. 22, 2008. Marker delivery device 10 may include a tubular elongate outer cannula 12 having a marker exit, such as side opening 14 formed near to, but spaced proximally from, the distal end of the cannula 12. FIGS. 4-9 illustrate various features of a biopsy marker delivery device of the type disclosed in copending, commonly assigned U.S. patent application Ser. No. 12/563,360 filed Sep. 21, 2009. FIGS. 10-15 illustrate one or more biopsy marker delivery devices according to the present invention.

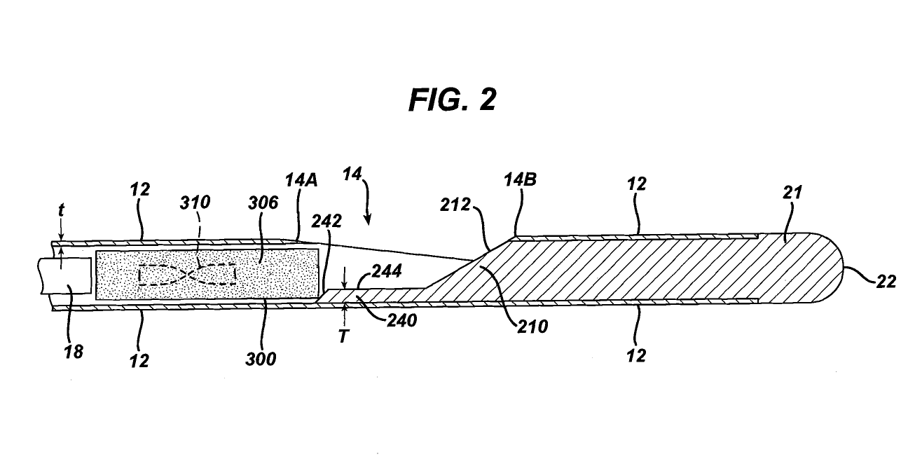

Referring to FIGS. 1-3, a grip 16 can be provided at the proximal end of cannula 12. A pushing member in the form of a push rod 18 can be provided, with push rod 18 extending coaxially in cannula 12 such that the push rod 18 is configured to translate within cannula 12 to displace one or more markers through the side opening 14 (see FIG. 2). Rod 18 can have a proximal portion (proximal portion 18A in FIG. 7) have sufficient rigidity in compression to push a marker from the internal lumen of cannula 12 out through opening 14, and include a more distal portion (for example portion 18B in FIG. 7) that is relatively flexible in bending so that the cannula 12 can be inserted along a curved path to deploy a marker element at a biopsy site.

A plunger 20 can be provided at the proximal end of rod 18 for forcing rod 18 distally in cannula 12 to deploy a marker out of the cannula 12. A user may grasp grip 16 with two fingers, and may push on plunger 20 using the thumb on the same hand, so that the marker delivery device 10 can be operated by a user's single hand. A spring (not shown) or other feature may be provided about rod 18 to bias rod 18 proximally relative to grip 16 and cannula 12.

FIG. 2 depicts a cross-sectional view of a distal portion of the marker delivery device 10. FIG. 2 shows a biopsy marker 300 disposed in the internal lumen 15 of the cannula 12. The marker 300 can comprise a biodegradable or otherwise resorbable body 306, such as a generally cylindrically shaped body of collagen, and a metallic, generally radiopaque marker element 310 (shown in phantom) disposed within or otherwise carried by the body 306.

The cannula 12 can be formed of any suitable metallic or non-metallic material. In one embodiment, the cannula 12 is formed of a thin walled hollow tube formed of a suitable medical grade plastic or polymer. One suitable material is a thermoplastic elastomer, such as Polyether block amide (PEBA), such as is known under the tradename PEBAX. The cannula 12 can be formed of PEBAX, and can be substantially transparent to visible light and X-ray.

The side opening 14 can be formed by cutting away a portion of the wall of cannula 12. The side opening 14 communicates with an internal lumen 15 of the cannula. The side opening 14 can extend axially (in a direction parallel to the axis of the lumen 15) from a proximal opening end 14A to a distal opening end 14B, as illustrated in FIG. 2.

The distal tip 22 extending from the distal end of cannula 12 can be rounded as shown in FIG. 2. Referring to FIG. 2, a marker delivery device can have the distal end of the cannula 12 closed by a unitary endpiece 21 formed in place in the distal end of the cannula 12, with a part of the endpiece 21 extending into the internal lumen 15 of the cannula. The distal endpiece 21 can be a molded or cast component, and can provide an integrally formed combination of the tip 22, a ramp 210 having a ramp surface 212, and a marker engaging element 240. The ramp surface 212 aids in directing the marker 300 from the internal lumen 15 through side opening 14. The marker engaging element 240 may be employed to retain the marker 300 in the internal lumen 15 until the user intends to deploy the marker.

The marker engaging element 240 may be disposed within the internal lumen 15, and at least a portion of the marker engaging element is disposed distally of the proximal end 14A of side opening 14. The marker engaging element 240 can extend along a portion of the floor of the cannula 15 under the opening 14, and the marker engaging element 240 can be positioned to reinforce the portion of the cannula in which the opening 14 is formed. For instance, by positioning the marker engaging element 240 underneath the opening 14, as shown in FIG. 2, the element 240 can help to stiffen the cannula 12 in the region where wall of the cannula 12 is cut to form the opening 14. In FIG. 2, the marker engaging element 240 extends from the proximal most portion of ramp surface 212, and does not extend proximally of the side opening 14, though in other embodiments, a portion of the element 240 could extend proximally of the opening 14.

In the embodiment shown in FIG. 2, marker engaging element 240 is in the form of a step having a generally uniform thickness T along the element's axial length, except that the element has a tapered proximal end 242. The tapered proximal end 242 can form an included angle with the longitudinal axis of the lumen 15 (included angle with a horizontal line in FIG. 2) of about 45 degrees, while the ramp surface 212 can form an included angle with the longitudinal axis of about 30 degrees.

The thickness T can be greater than the wall thickness t of the cannula 12, and in one embodiment T is at least about twice the thickness t. In one embodiment, the thickness T can be between about 0.018 inch to about 0.040 inch, and the wall thickness t can be between about 0.005 inch to about 0.008 inch. The internal diameter of lumen 15 can be about 0.120 inch.

In FIG. 2, the upwardly facing surface 244 (surface facing the opening 14) marker engaging element 240 extends distally to contact the ramp surface 212, so that there is not a space or gap between the surface 244 and the ramp surface 212. Such an arrangement is advantageous to reduce the possibility that the marker 300, upon moving past the marker engaging element, will become lodged between the marker engagement element and the ramp.

If desired, the marker engaging element 240, ramp 210, and/or the tip 22 can be formed of, or include, a material that is relatively more radiopaque than the wall of the cannula 12. For instance, where the element 240, ramp 210, and tip 22 are formed as an integral endpiece 21, the endpiece 21 can include a radiopaque additive, such as barium sulfate. For instance, the endpiece 21 can be a component molded of PEBAX, with about 20 percent by weight barium sulfate added to the molten PEBAX mold composition.

The relatively more radiopaque marker engaging element 240, ramp 210, and tip 22 can be useful in distinguishing the position of those components using radiographic imaging. Also, where the ramp and/or step of engaging element are positioned in association with the opening 14, the addition of a radiopaque material can help identify the position of the opening, and the position of the marker 300 relative to the opening before, during, or after deployment of the marker.

Only one marker is shown disposed in lumen 15 in the figures. However, it will be understood that multiple markers can be disposed in marker delivery device 10, such as in an end to end configuration. The markers can have the same size and shape, or alternatively have different sizes and/or shapes.

The cannula 15 can be generally transparent to visible light and x-ray, and the endpiece 21 can be generally opaque to visible light and x-ray. If desired, the endpiece 21 can be colored with a dye or other suitable colorant in the liquid mold composition. For example, it may be desirable to have different size markers (e.g. length and/or diameter) for different biopsy procedures. For instance, it may be desirable to provide a larger marker if a relatively large biopsy sample is taken, and a smaller marker if a relatively small biopsy sample is taken. The endpiece 21 can be colored using one of multiple colors to indicate the size of the marker disposed in the cannula. For instance, if three marker sizes are provided, the endpiece 21 can be colored one of three colors to identify which of the marker sizes are disposed in the cannula of a particular marker device. The endpiece 21 can also be colored to indicate a particular size (diameter or length) biopsy needle with which the marker delivery device is to be used. Additionally, multiple marker delivery devices could be packaged in kit form, with the kit including marker delivery devices having different size markers and correspondingly colored end pieces.

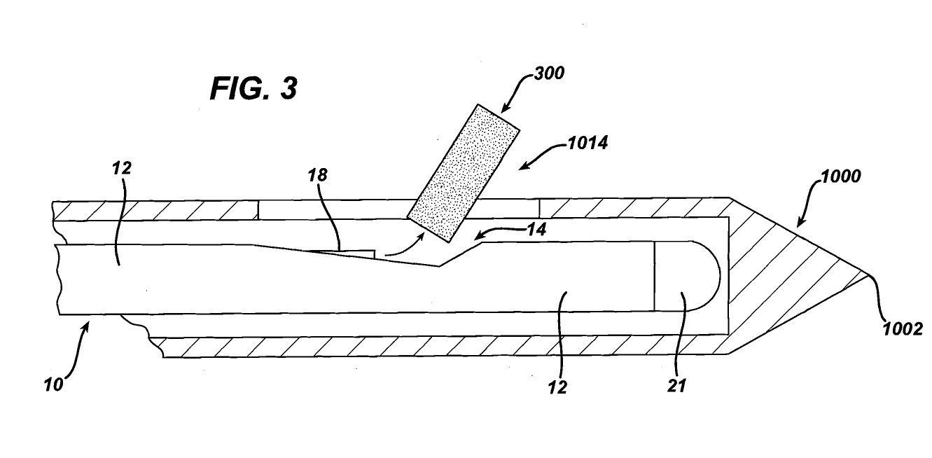

Referring to FIG. 3, the marker delivery device 10 may be used to deploy a marker to mark a particular location within a patient. In FIG. 3, a cannular biopsy needle 1000 is shown. The needle 1000 is shown having a closed distal end with piercing tip 1002, and a lateral tissue receiving aperture 1014. Marker deployer 10 may be introduced to a biopsy site through biopsy needle 1000, which can be the same needle used to collect a tissue sample from the biopsy site. The biopsy needle 1000 can be of the type used with single insertion, multiple sample vacuum assisted biopsy devices. Several such biopsy devices are disclosed in the various patents and patent applications that have been referred to and incorporated by reference herein, though other biopsy devices may be used.

FIG. 3 shows the distal end of a marker deployer 10 disposed within the needle 1000. The needle 1000 can be positioned in tissue, and a biopsy sample can be obtained through opening 1014, thereby providing a biopsy cavity adjacent opening 1014. Then, after the tissue sample has been obtained and transferred proximally through the needle, and without removing the needle 1000 from the patient's tissue, the deployer 10 can be inserted into a proximal opening in the needle 1000. In FIG. 3, the needle 1000 and deployer 10 are positioned such that opening 14 of cannula 12 and opening 1014 of needle 1000 are substantially aligned axially and circumferentially. Then, with the deployer and needle so positioned at the biopsy site, the push rod 18 can be advanced to deploy the marker up the ramp surface 212, through the opening 14, and then through opening 1014, into the biopsy cavity.

In some instances, it may be necessary to bend or otherwise flex the marker deployer cannula 12 and push rod 18 when inserting the deployer into the biopsy device. By reducing the effective contact surface area between the outer surface of the push rod 18 and the inner surface of the cannula 12, Applicants believe the tendency of the push rod 18 to “lock” within the cannula 12 can be reduced and/or eliminated.

Referring now to FIGS. 4-9, FIG. 4 illustrates a portion of the cannula 12 and push rod 18, with part of the cannula 12 cut away to show the push rod 18 disposed within the cannula 12. The cannula 12 can be formed from a thin wall, flexible non-metallic tube having a generally smooth outer surface 124, a generally smooth inner surface 122, and having an inner diameter designated 126 in FIG. 4. A generally flexible, elongate pushing member, such as a portion of push rod 18, is disposed at least partially within the internal lumen of the hollow cannula 12. The push rod 18 has an outer diameter designated 186 in FIG. 4.

In FIG. 4, push rod 18 is illustrated having an outer surface 182 that has a surface feature designated generally as 184, which surface feature is effective for reducing the contact surface area between the outer surface of the push rod 18 and the inner surface of the lumen extending through cannula 12 when the cannula 12 and rod 18 are bent or otherwise flexed. In one embodiment, the surface feature 184 is configured to be effective in providing at least about a 50 percent reduction (still more particularly at least about 75% reduction) in the contact surface area that would otherwise occur for a push rod 18 and cannula 12 both having generally smooth, untextured surfaces and the same nominal outer diameter and inner diameter.

In the embodiment shown in FIG. 4, surface feature 184 is shown comprising a plurality of longitudinally extending elevated portions in the form of ribs 188. The ribs 188 extend along at least a portion of the push rod 18 disposed within cannula 12.

For marker deployers 10 useful in connection with breast biopsy devices having a breast biopsy needle, and useful for deploying breast biopsy markers from breast biopsy devices, the inner diameter 126 of the lumen of cannula 12 may be (but is not limited to) at least about 0.08 inch, and the outer diameter 186 of the push rod 18 may be (but is not limited to) between about 0.04 inch and about 0.09 inch.

In one embodiment, the ribs 188 can have a radial height 196 measured with respect to adjacent recessed portions (designated as valleys 189) of between about 0.0001 inch and about 0.01 inch. More particularly, the ribs 188 can have a radial height of between about 0.0003 inch and about 0.004 inch, yet more particularly, the radial height 196 can be between about 0.0005 inch and about 0.004 inch. In one non-limiting example, the radial height 196 can be between about 0.001 inch and about 0.003 inch, such as about 0.002 inch plus or minus 0.001 inch. The radial height 196 can be less than one tenth of the diameter 186 of the push rod, and more particularly less than about one twentieth of the diameter 186. The radial height 196 can be less than one half (less than 50 percent of), and more particularly less than about one quarter of the difference between outer diameter 186 and the inner diameter 126 of the lumen of the cannula 12.

The number and size of longitudinal surface features may be selected to be effective in reducing the effective contact surface area between push rod and the inner surface of the cannula, without interfering with sliding of the push rod within the lumen of the cannula. For instance, but without being limited by theory, in one embodiment the push rod 18 may have at least about 20 ribs spaced around it's circumference, and less than about 100 ribs. The ribs can be formed by extruding, molding, or other suitable methods. The circumferential spacing between adjacent ribs can be greater than the radial height 196 of the adjacent ribs.

In one non limiting example, a biopsy marker deployer 10 of the present invention suitable for use through an 11 gauge breast biopsy needle can have a push rod diameter 186 of about 0.060 inch, a cannula inner diameter 126 of about 0.084 inch, and about 40-50 splines spaced around the circumference of the push rod, the splines being generally uniformly spaced apart and having a radial height of about 0.002 inch. Without being limited by theory, it is believed that such a configuration can be effective in reducing the effective contact area between the cannula 12 and the rod 18 to about 0.246 square inch from about 1.158 square inch.

In another non limiting example, a biopsy marker deployer 10 of the present invention suitable for use in an 8 gauge breast biopsy needle can have a push rod diameter 186 of about 0.082 inch, a cannula inner diameter 126 of about 0.120, and about 50-70 splines spaced around the circumference of the push rod, the splines being generally uniformly spaced apart and having a radial height of about 0.002 inch. Without being limited by theory, it is believed that such a configuration can be effective in reducing the effective contact area between the cannula 12 and the rod 18 to about 0.388 square inch from about 1.665 square inch.

FIG. 7 illustrates a push rod 18 having a relatively stiff proximal section 18A, and a flexible portion 18B comprising a plurality of ribs 188 as described above. The relatively stiff proximal portion 18A can comprise a metallic sleeve or other stiffening member disposed at the proximal end of the rod to prevent the proximal end of the push rod from bending or kinking when plunger 20 is pressed to deploy a marker. The flexible portion 18B may comprise ribs 188 or other surface features along some or substantially all the length of flexible portion 18B such as to be effective in preventing locking of the push rod 18 within cannula 12 when the rod and cannula are bent or otherwise disposed along a curved path.

FIG. 8 illustrates surface features 1188 disposed at spaced apart locations along the length of the push rod 18. The surface features 1188 may be in the form of longitudinally spaced apart raised rings extending circumferentially around the diameter of push rod 18. The rings may be circumferentially continuous or formed of discrete segments. In yet another alternative embodiment, the outer surface of the push rod 18 may comprise surface features in the form of bumps or protrusions, such as bumps or protrusions having the radial height characteristics set forth above. The bumps or protrusions may be randomly positioned on the surface of the rod 18, or may be arranged in a predetermined pattern.

FIG. 9 illustrates the cannula 12 having an inner surface 122A having a surface feature effective for reducing binding/locking of the rod 18 within the cannula 12.

FIGS. 10-15 illustrate a biopsy marker delivery device according to the present invention. In FIG. 10, a biopsy marker delivery device is illustrated including a tubular elongate outer cannula 12, a marker exit, such as a side marker exit 14 formed in a sidewall of cannula 12, a grip 16, a push rod 18, and a plunger 20. In addition, the biopsy marker delivery device in FIG. 10 includes a positioning component 400.

The positioning component 400 is shown disposed on the outer surface of the cannula sidewall, and is positioned along the length of the cannula between the proximal end of the cannula and the marker exit 14. In FIG. 10, the component 400 is shown positioned around the outer surface of cannula 12 at a longitudinal position intermediate marker exit 14 and grip 16. The component 400 is sized and shaped to limit the depth to which cannula 12 may be inserted into a biopsy instrument, such as a single insertion, multiple sample vacuum assisted biopsy device.

In one embodiment, the component 400 is formed on the cannula 12, such as by molding, to be stationary with respect to the cannula, and such that the component is positioned a predetermined fixed distance from the distal tip 22 of endpiece 21 and the marker exit 14.

FIG. 11 provides a schematic, enlarged view of a distal portion of the biopsy marker delivery device in FIG. 10. As shown in FIG. 11, the marker exit may optimally include a relatively smaller proximal portion, and a relatively enlarged distal portion 145.

FIG. 12 provides a schematic illustration of a portion of the biopsy marker delivery device (including component 400) positioned with respect to a proximal end 910 of a biopsy device 900. Biopsy device 900 may be a biopsy device of the type including a needle 1000 (FIG. 3). For instance, biopsy device 900 may be a single insertion, multiple sample vacuum assisted biopsy device. Cannula 12 may be sized to fit through a proximal opening in the proximal end 910 of biopsy device 900, and component 400 may be sized and shaped to ensure that the cannula 12 is not over inserted into the device 900. For instance, the component 400 may be sized and shaped to ensure that the end piece 21 of the marker delivery device does not extend out of needle 1000 of biopsy device 900.

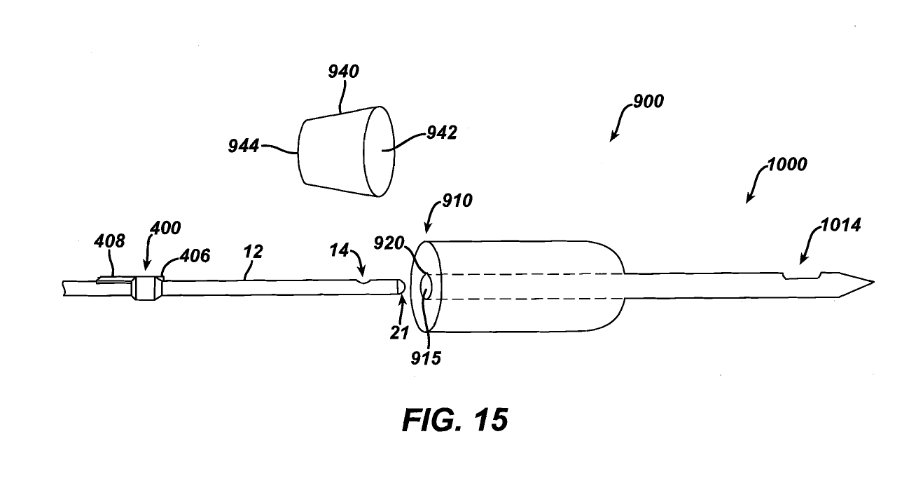

FIG. 15 illustrates schematically how the marker delivery device having a component 400 may be inserted through a proximal opening 915 at a proximal portion 910 of a biopsy device 900 having needle 1000. The marker delivery device may be inserted through opening 915 and advanced within biopsy device 900, with component 400 permitting the exit 14 to be registered with needle opening 1014 (as seen in FIG. 3), while component 400 prevents over insertion of the cannula 12 in biopsy device 900. In FIG. 15, a proximal cover 940 is shown removed from proximal portion 910 to expose opening 915. Opening 915 may communicate directly or indirectly with the lumen of needle 1000. Cover 940 may be in the form of a cup or tissue sample holder having an open distal end 942 and a closed proximal end 944, the cover 940 being releasable with respect to the proximal portion 910, and the cover 940 covering opening 915 during biopsy sampling.

Referring to FIGS. 10-15, the component 400 may include a generally annular shaped body 402. The component 400 may also include a proximally extending portion, such as a proximally extending tab 408 shown in FIGS. 10 and 12. The proximally extending tab 408 is shown to be aligned circumferentially with respect to (generally same o'clock position as) the marker exit 14. The annular body 402 can include a distally facing tapered surface 404, and an alignment feature, such as a distally extending male alignment feature 406 that engages with a female alignment feature 920 of the biopsy device 900. Engagement of feature 406 with feature 920 helps maintain alignment of marker exit 14 with needle side opening 1014.

As shown in FIG. 14, the positioning component 400 is spaced a predetermined axial (longitudinal) distance 403 from the distal tip 22 of end piece 21. The distance 403 may be provided by overmolding the component 400 on the outer surface of the cannula 12.

In one embodiment, the component 400 and the end piece 21 may be formed together, such as by molding the component 400 over the cannula 12 and molding end piece 22 in the distal end of cannula 12 in the same mold and during the same mold operation. For instance, the hollow tube may be placed within a mold having a mold cavity corresponding to the component 400 and a mold cavity corresponding to the end piece 21.

The biopsy marker delivery device may be formed by obtaining a thin wall, flexible tube for use as cannula 12, forming the end piece 21 in the distal end of the tube, such as by molding, to close the distal end of the tube; overmolding the positioning component 400 on the outer surface of the hollow tube a predetermined distance from the distal end of the tube; such that the component 400 is positioned along the tube at a predetermined distance from the distal tip 22 of the end piece 21, and positioned between the proximal end of the hollow tube and the distal end of the hollow tube. The marker exit 14 may be formed by cutting or otherwise forming an opening in the sidewall of the tube. After the end piece 21 and the component 400 are molded on the tube, at least marker may be positioned with the lumen of the tube through the proximal end of the tube.

In another embodiment, the component 400 may be formed separately, and then slid over cannula 12. In such an embodiment, the component 400 may be fixed with respect to the cannula 12, or alternatively, be sized to slide along the length of the cannula 12 so that the component 400 may be positioned at one or more axial markings or indicia spaced along the length of the tube. For instance, the cannula 12 may have a plurality of axial markings along its length, each marking associated with a different size/length of biopsy needle 1000. The user may then slide the component 400 to the desired indicia corresponding to the biopsy device 900/needle 1000 into which the cannula 12 is to be inserted, thereby ensuring that the cannula 12 is inserted to the correct depth with respect to the biopsy device 900/needle 1000 being used.

The component 400 may be sized such annular body 402 has an axial length of less than about 1 inch, and more particularly less than about 0.5 inch. The proximally extending tab 408 can have a length at least about twice the axial length of the annular body 402. In one non-limiting embodiment, the axial length of the body 402 can be about 0.2 to about 0.3 inch, the outer diameter of body 402 can be about 0.15 inch to about 0.16, the inner diameter of body 402 can be about 0.09 to about 0.11 inch, and the combined axial length of body 402 and tab 408 can be about 0.7 to about 0.8 inch.

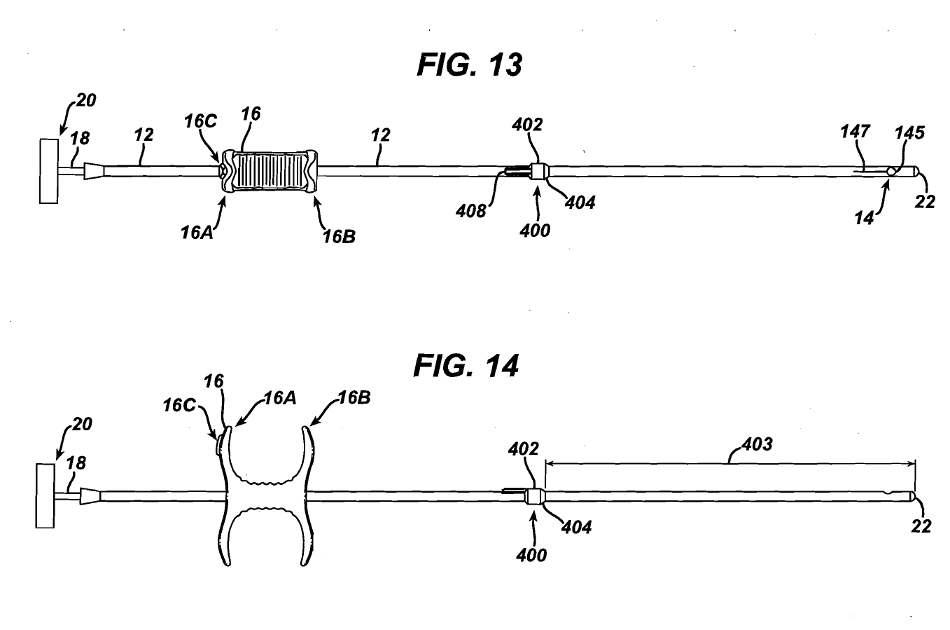

Referring to FIGS. 13 and 14, the biopsy marker delivery device may have a marker exit 14 comprising a proximal portion in the form of a narrow opening or slit 147, and a distal portion in the form of a circular or oval opening 145. The proximal tab 408 may be aligned with the exit 14, as well as with finger extensions 16A and 16B of grip 16. An orientation mark, such as an arrow shaped raised portion 16C on finger extension 16A, may also be aligned with proximally extending tab 408.

If desired, grip 16 and/or positioning component 400 and/or end piece 21 may be formed of the same material (such as a non-metallic material) and/or have the same color, such as where the color is indicative of a particular gauge size needle into which the marker delivery device is meant to be inserted. In one embodiment, the component 400 and end piece 21 are molded (such as by being molded together in the same mold and/or during the same molding operation) using the same material and with the same color, depending on the needle gauge for which the biopsy marker delivery device is intended to be used. By way of example, the component/end piece could be molded with a green material for 8 gauge applications, a blue color for 11 gauge applications, and a red color for 14 gauge applications. Additionally, a kit may be provided having multiple marker delivery devices packaged together, at least some of the marker delivery devices being sized to be inserted in different gauge needles 1000.

In another embodiment, the end piece 21 and/or the component 400 may be attached to the cannula 12, such as by an adhesive, by heat bonding, ultrasonic welding, or other suitable methods.

The present invention has been disclosed with respect to a biopsy marker deployer device. However, the various features and components disclosed in the figures may be employed in devices useful with radioisotope applications, as in PEM, BSGI, and other imaging methods that may employ a radioisotope or other radiation source in connection with imaging a biopsy procedure.

Embodiments of the devices disclosed herein are generally designed to be disposed of after a single use, but could be designed to be used multiple times. After forming the marker, and inserting the marker into the deployer, the biopsy device can be sterilized. The device can be placed in a package, such as plastic or TYVEK bag.

The packaged biopsy device may then be placed in a field of radiation such as gamma radiation, x-rays, or high-energy electrons to sterilize the device and packaging. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. Accordingly, the scope of the present invention should be considered in terms of the following claims and is understood not to be limited to the details of structure and operation shown and described in the specification and drawings.

What is claimed:

1. A method of forming a biopsy marker delivery device, the method comprising the steps of:
obtaining a hollow tube having a sidewall, a proximal end, a distal end, and an internal lumen;
forming an endpiece in place in the distal end of the hollow tube to close the distal end of the tube;
forming a component extending around an outside surface of the hollow tube, wherein the step of forming the component comprises positioning the component along the tube at a predetermined distance from the endpiece between proximal end of the hollow tube and the distal end of the hollow tube; and
disposing at least one biopsy marker in the internal lumen.

2. The method of claim 1 further comprising forming an opening in the side wall of the tube between the distal end of the hollow tube and the component.

3. The method of claim 1 wherein the component substantially encircles the hollow tube.

4. The method of claim 1 wherein the component and endpiece comprise a non-metallic material.

5. The method of claim 1 wherein the step of forming the component comprises molding the component on the hollow tube.

6. The method of claim 1 wherein the component and the endpiece are formed on the tube at substantially the same time.

7. The method of claim 1 wherein the component and the endpiece are molded to the hollow tube in a single molding operation.

8. The method of claim 1 wherein the step of forming the component comprises forming a unitary component comprising an annular body having a distally facing tapered surface.

9. The method of claim 1 wherein the step of forming the component comprises forming a unitary component having an annular body and a proximally extending tab.

10. A biopsy marker delivery device for use with a biopsy instrument, the biopsy marker delivery device comprising:
a cannula having an internal lumen extending from a proximal end of the cannula to the distal end of the cannula, and a marker exit formed in a sidewall of the cannula proximal to the distal end of the cannula;
a distal tip closing the distal end of the cannula;
a component disposed on the outer surface of the cannula sidewall, the component disposed at a position along the length of the cannula between the proximal end of the cannula and the marker exit formed in the sidewall of the cannula, and the component being sized and shaped to limit the depth to which the cannula can be inserted within a biopsy instrument; and
at least one marker disposed within the internal lumen of the cannula.

11. The device of claim 10 wherein the component is disposed at a position along the length of the cannula at a predetermined distance from the distal tip.

12. The device of claim 10 wherein the component comprises an annular body and a distally facing tapered surface.

13. The device of claim 10 wherein the component is overmolded on the cannula.

14. The device of claim 10 wherein the component comprises an annular body and a proximally extending tab.

15. The device of claim 14 wherein the proximally extending tab is circumferentially aligned with the marker exit port.

16. A biopsy marker delivery device comprising:
a cannula having a proximal end, a distal end, an internal lumen extending from the proximal end to the distal end, and a side marker exit port disposed proximally of the distal end of the cannula;
a distal tip closing the distal end of the cannula;
a grip supported adjacent the proximal end of the cannula;
a component disposed around the cannula and positioned between the side marker exit port and the grip; and
at least one marker disposed within the internal lumen of the cannula.

17. The biopsy marker device of claim 16 wherein the component is positioned on the outer surface of the cannula at a predetermined distance from the distal tip.

18. The biopsy marker device of claim 16 wherein the component comprises an annular body having a distally facing tapered surface.

19. The biopsy device of claim 16 wherein the component comprises a proximally extending tab aligned with the exit port.

20. The biopsy device of claim 19 wherein the component comprises an annular body having an axial length of less than about 0.5 inch and an axially extending tab, the tab extending proximally from the annular body, and the tab having a length at least about twice the axial length of the annular body.
